(19) United States
(12) Patent Application Publication
(10) Pub. No.: US 2014/0322773 A1
(43) Pub. Date: Oct. 30, 2014

(54) METHOD FOR PRODUCING LACTIC ACID

(71) Applicant: Asahi Glass Company, Limited, Tokyo (JP)

(72) Inventors: Futoshi HARA, Tokyo (JP); Hideki Tohda, Tokyo (JP)

(73) Assignee: Asahi Glass Company, Limited, Tokyo (JP)

(21) Appl. No.: 13/971,512

(22) Filed: Aug. 20, 2013

Related U.S. Application Data
(63) Continuation of application No. PCT/JP2012/053709, filed on Feb. 16, 2012.

(30) Foreign Application Priority Data
Feb. 21, 2011 (JP) 2011-035165

(51) Int. Cl. C12P 7/56

(52) U.S. Cl. CPC C12P 7/56
USPC 435/139; 435/254.2

(57) ABSTRACT

Provided is a method for producing lactic acid which does not require neutralization and crude purification associated therewith both of which give a high load to the environment.

A method for producing lactic acid, which comprises subjecting glucose to lactic acid fermentation by using a fission yeast having a lactic acid fermentation ability and recovering lactic acid produced thereby, characterized in that a fermentation liquor created from an aqueous glucose solution by lactic acid fermentation is replaced with an aqueous glucose solution having a potassium ion concentration of at least 400 ppm to continue the lactic acid fermentation, and the replacement of the fermentation liquor with the potassium ion-containing aqueous glucose solution is performed at least once.

### TECHNICAL FIELD

The present invention relates to a method for producing lactic acid, and specifically, relates to a method for producing lactic acid by using a fission yeast having a lactic acid fermentation ability.

### BACKGROUND ART

Lactic acid is one of hydroxy acids, and is also referred to as 2-hydroxy propanoic acid. L-lactic acid, which is one of its isomers, is generated by the glycolytic system of various organisms including mammals and microbes, and is abundant in nature.

In recent years, it has been attracted attention that polylactic acid comprised of lactic acids linked by an ester bond between carboxyl group and hydroxy group can be produced from a component derived from biomass, and is a plastic which is biodegradable by microbes present in the ground or the like. Therefore, commercialization of polylactic acid has been attempted in various forms including polylactic acid itself, a polymer alloy made of polylactic acid and other resins, etc.

For the production of lactic acid, a method of utilizing a lactic acid fermentation by microbes as represented by a lactic acid bacterium is used. For example, Patent Document 1 discloses a lactic acid fermentation by Lactobacillus delbrueckii (L. delbrueckii) which is one of lactic acid bacteria, Non-Patent Document 1 discloses a lactic acid fermentation by Corynebacterium glutamicum (C. glutamicum) which is one of actinomycetes, Non-Patent Document 2 discloses a lactic acid fermentation by a budding yeast Saccharomyces cerevisiae (S. cerevisiae), and Non-Patent Document 3 discloses a lactic acid fermentation by a yeast of the genus candida, Candida utilis (C. utilis).

However, all the organisms used in the above-described Non-Patent Documents are weak against acids, and their lactic acid fermentation abilities decrease significantly when lactic acids accumulate as the fermentation proceeds and the pH of a culture broth becomes low, whereby neutralization by calcium carbonate, etc. becomes necessary. As a result, in addition to the generation of a large amount of carbon dioxide at the time of the neutralization, when recovering lactic acid from the culture broth containing cells after the lactic acid fermentation, there has been a problem such that a crude purification is required to be carried out for removing the precipitated calcium sulfate by adding sulfuric acid to calcium lactate generated by the neutralization and producing lactic acid and calcium sulfate (gypsum).

As the method for producing lactic acid without carrying out neutralization with an alkali, a method of using an acid resistant microorganism such as a yeast of the genus Saccharomyces as a host to prepare a transformant by introducing a gene encoding a lactate dehydrogenase into the acid resistant microorganism (Patent Document 2), and a method of using Saccharomyces cerevisiae (budding yeast) in which a gene encoding a lactate dehydrogenase is introduced and a gene encoding pyruvate decarboxylase 1 is deleted or inactivated (Patent Document 3) have been known.

### PRIOR ART DOCUMENTS

### Patent Documents

Patent Document 1: US-A-2007/0212765
Patent Document 2: JP-A-2001-204464
Patent Document 3: JP-A-2008-48726

### Non-Patent Documents

Non-Patent Document 1: Okino et al., Applied microbiology and biotechnology, 2005 September; 68 (4): 475-80.
Non-Patent Document 2: Saitoh et al., Applied and environmental microbiology, 2005 May; 71 (5): 2789-92.
Non-Patent Document 3: Ikushima et al., Bioscience, biotechnology, and biochemistry, 2009 August; 73 (8): 1818-24

### DISCLOSURE OF INVENTION

### Technical Problem

However, the method described in Patent Document 2 can provide merely from 2 to 5% of lactic acid even after culturing for from 20 to 24 hours, and therefore its productivity is not sufficient. Further, the method described in Patent Document 3 is not suitable for the industrial mass production of lactic acid since it requires neutralization with an alkali for producing lactic acid in a large amount. Accordingly, development of a method which can produce lactic acid with high productivity without carrying out neutralization with an alkali has been desired.

The present inventors focused on the fact that a fission yeast as represented by Schizosaccharomyces pombe has high resistance to acids and does not require neutralization, and have found that the above-mentioned problems can be solved by carrying out lactic acid fermentation by using a fission yeast having a lactic acid fermentation ability.

Further, to produced lactic acid by using the fission yeast having a lactic acid fermentation ability, the present inventors have examined lactic acid fermentation of the fission yeast which uses glucose as a carbon source. The nutrient-rich broth such as a yeast complete broth for growing fission yeast is not suitable for lactic acid fermentation since it contains components not necessary for lactic acid fermentation in a large amount, and removal of such components as impurities is required when recovering lactic acid after fermentation. Therefore, they examined, as the culture broth for lactic acid fermentation, an aqueous solution which contains glucose as the carbon source and does not contain components not necessary for lactic acid fermentation as much as possible (or contains in a small amount).

In the present specification, culture broth for growing fission yeast is referred to as growing culture broth, culture broth to be used for lactic acid fermentation is referred to as fermentation culture broth, and culture broth in which lactic acid is accumulated by carrying out lactic acid fermentation continuously to some extent is referred to as fermentation liquor, to distinguish each of them.

The growing culture broth is the above described nutrient-rich broth or the like, and is a culture broth for increasing the cell number by growing the fission yeast. At the time of growing the fission yeast, lactic acid fermentation proceeds to some extent and lactic acid may be produced. However, such lactic acid fermentation is not intended for the production of lactic acid.

The fermentation culture broth is an aqueous solution containing glucose, and hereinafter sometimes referred to as aqueous glucose solution. The fermentation culture broth may contain a carbon source other than glucose, but preferably contains organic nutrient sources other than carbon source (e.g. nitrogen source) as less as possible. It is preferred that inorganic nutrients sources required for lactic acid fermentation is contained therein. During lactic acid fermentation, the fission yeast may grow to some extent. However, such lactic acid fermentation is not intended for the growth of the fission yeast.

The culture broth used for lactic acid fermentation contains the fission yeast, but the above fermentation liquor is a portion other than the fission yeast. The fermentation liquor contains lactic acid, and may further contain a carbon source such as unfermented glucose remained therein. Further, it may contain ethanol since ethanol fermentation occurs sometimes along with lactic acid fermentation.

In order to increase the efficiency of lactic acid fermentation, it is preferred to accumulate lactic acid in the fermentation liquor as much as possible, and continue the lactic acid fermentation until the amount of glucose remained therein becomes low. Further, it is preferred that the fission yeast separated after completion of lactic acid fermentation are used repeatedly for carrying out lactic acid fermentation in a fresh fermentation culture broth. Further, the lactic acid fermentation may be continued successively. That is, the lactic acid fermentation may be continued by separating a part of the fermentation liquor successively while carrying out lactic acid fermentation and, at the same time, supplying the fermentation culture broth. Further, the lactic acid fermentation may be continued by separating a part of the fermentation liquor intermittently and, at the same time, supplying the fermentation culture broth.

The present inventors conducted so-called repetitive fermentation by separating the fission yeasts after lactic acid fermentation and subjecting them to lactic acid fermentation in a fresh fermentation culture broth, and found that the lactic acid fermentation ability of the fission yeast decreases significantly at the stage where the fermentation culture broth is replaced once to several times. Such a decrease in the lactic acid fermentation ability is considered to occur also in serial fermentation when the amount of a fermentation culture broth supplied becomes large.

### Solution to Problem

The present inventors have conducted extensive studies to solve the above-mentioned problems, and as a result, have found that it is possible to suppress the decrease in the lactic acid fermentation ability of the fission yeast by introducing a potassium ion into a fermentation culture broth.

In a case where the repetitive fermentation is carried out by using pre-grown fission yeast, the decrease in the lactic acid fermentation ability is not observed even after the fermentation culture broth is replaced several times from the beginning. From this, in lactic acid fermentation, it is supposed that the potassium component in the cells of fission yeasts are gradually leaked into the fermentation liquor and is lost from the fermentation system by replacing the fermentation culture broth so that the leaked potassium component is not re-absorbed by the cells, whereby the decrease in the lactic acid fermentation ability occurs when the amount of the potassium component in the cells decreases and reaches to a certain limit value. Therefore, in the repetitive fermentation, the decrease in lactic acid fermentation ability can be suppressed by using a fermentation culture broth containing a potassium ion in a certain amount or higher as the fermentation culture broth for replacing, before the lactic acid fermentation ability of the fission yeast decreases.

The present invention has been accomplished based on the above-mentioned findings, and relates to a method for producing lactic acid by using a fission yeast having a lactic acid fermentation ability and a fermentation activator which are shown below as [1] to [15].

[1] A method for producing lactic acid, which comprises subjecting glucose to lactic acid fermentation by using a fission yeast having a lactic acid fermentation ability and recovering lactic acid produced thereby, characterized in that a fermentation liquor created from an aqueous glucose solution by lactic acid fermentation is replaced with an aqueous glucose solution having a potassium ion concentration of at least 400 ppm to continue the lactic acid fermentation, and the replacement of the fermentation liquor with the potassium ion-containing aqueous glucose solution is performed at least once.
[2] The method for producing lactic acid according to [1], wherein a fermentation liquor created from the aqueous glucose solution having a potassium ion concentration of at least 400 ppm by lactic fermentation is replaced with an aqueous glucose solution having a potassium ion concentration of lower than 400 ppm at least once.
[3] The method for producing lactic acid according to [1] or [2], wherein the growth rate of the fission yeast as represented by the following equation is at most 1.5.

Growth rate=(dried cell weight after fermentation for 7 hours)/(dried cell weight at the time of starting fermentation)

[4] The method for producing lactic acid according to any one of [1] to [3], wherein the aqueous glucose solution to be used for the lactic acid fermentation contains from 30 to 200 g/L of glucose.
[5] The method for producing lactic acid according to any one of [1] to [4], wherein the potassium ion concentration of the aqueous glucose solution having a potassium ion concentration of at least 400 ppm is at most 4,000 ppm.
[6] The method for producing lactic acid according to any one of [1] to [5], wherein the aqueous glucose solution to be used for the lactic acid fermentation contains at least one type of a metal ion selected from the group consisting of alkali metal ions other than a potassium ion and alkali earth metal ions.
[7] The method for producing lactic acid according to any one of [1] to [6], wherein the aqueous glucose solution to be used for the lactic acid fermentation has a nitrogen source content of from 0 to 3.0 g/L.
[8] The method for producing lactic acid according to any one of [1] to [7], wherein the aqueous glucose solution to be used for the lactic acid fermentation does not contain ions of metals which are other than alkali metals and alkali earth metals and which are required for the growth of fission yeast, or does not contain such ions in an amount necessary for the growth of fission yeast.
[9] The method for producing lactic acid according to any one of [1] to [3], wherein the aqueous glucose solution having a potassium ion concentration of at least 400 ppm contains from 50 to 150 g/L of glucose, from 400 to 4,000 ppm of potassium ion, at least one type of a metal ion selected from the group consisting of alkali metal ions other than a potassium ion and alkali earth metal ions, an anion which is a counter ion of the metal ion including potassium ion, from 0 to 300 ppm of a micronutrient source other than the above-mentioned ones, and from 0 to 300 ppm of a nitrogen source (when the anion and the micronutrient source contain nitrogen atoms, the amount of such nitrogen atoms is included).
[10] The method for producing lactic acid according to any one of [1] to [9], wherein the lactic acid fermentation is carried out by using cells which are collected after culturing and growing a fission yeast having a lactic acid fermentation ability in a liquid culture broth.
[11] The method for producing lactic acid according to [10], wherein the first lactic acid fermentation using the grown cells is carried out by using an aqueous glucose solution containing from 30 to 200 g/L of glucose.
[12] The method for producing lactic acid according to [11], wherein the aqueous glucose solution to be used for the first lactic acid fermentation does not have a potassium ion concentration of at least 400 ppm.
[13] The method for producing lactic acid according to any one of [1] to [12], wherein the fission yeast having a lactic acid fermentation ability is a transformant expressing a gene encoding a lactate dehydrogenase derived from an organism excluding fission yeast.
[14] The method for producing lactic acid according to any one of [1] to [13], wherein the fission yeast having a lactic acid fermentation ability is a transformant in which pdc2 gene of fission yeast is deleted or inactivated.
[15] A fermentation activator for activating a lactic acid fermentation by a fission yeast having a lactic acid fermentation ability in an aqueous glucose solution having a nitrogen source content of at most 0.3 g/L, characterized in that it is comprised of a water-soluble potassium compound which can generate a potassium ion.

### Effects of Invention

According to the present invention, it is possible to provide a method for producing lactic acid which does not require neutralization and crude purification associated therewith both of which give a high load to the environment.

Further, according to the present invention, it is possible to provide a fermentation activator for activating a lactic acid fermentation by a fission yeast having a lactic acid fermentation ability in an aqueous glucose solution having a nitrogen source content of at most 0.3 g/L.

### DESCRIPTION OF EMBODIMENTS

Heretofore, when subjecting a saccharide as a carbon source to lactic acid fermentation by using a microorganism having a lactic acid fermentation ability to produce lactic acid, an aqueous saccharide solution having an inorganic nutrient source added therein may sometimes have been used as the aqueous saccharide solution for lactic acid fermentation. However, adjustment of the amount of a certain inorganic substance by focusing on the specific inorganic substance has not been attempted. When using the aqueous saccharide solution having an inorganic nutrient source added therein, compounds containing potassium may sometimes have been used as the inorganic nutrient source. However, attention to potassium has not been paid, and the potassium ion concentration of the aqueous saccharide solution has been at most around 100 ppm. In the present specification, ppm means mg/(water 1 kg).

The method for producing lactic acid by using the aqueous glucose solution of the present invention is characterized in that an aqueous glucose solution containing potassium ion in a certain amount or higher is used as at least a part of the aqueous glucose solution (fermentation culture broth). Further, the present invention is characterized by continuing lactic acid fermentation by replacing the aqueous glucose solution used for the fermentation with a fresh aqueous glucose solution.

As described above, when repetitive fermentation is carried out by using pre-grown fission yeast, the decrease in the lactic acid fermentation ability of the fission yeast may sometimes not be observed even after the aqueous glucose solution is replaced several times from the beginning. The aqueous glucose solution used here is an aqueous glucose solution having a potassium ion concentration of at most around 100 ppm like one used in a conventional lactic acid fermentation, and is usually an aqueous glucose solution having a lower potassium ion concentration than that. Hereinafter, an aqueous glucose solution having such a low potassium ion concentration (i. e. lower than 400 ppm) is referred to as low-K aqueous glucose solution. The potassium ion concentration of the low-K aqueous glucose solution may be 0 ppm. Further, an aqueous glucose solution having a potassium ion concentration of at least 400 ppm, preferably from 400 to 4,000 ppm is referred to as high-K aqueous glucose solution. Unless specifically mentioned, these low-K aqueous glucose solution and high-K aqueous glucose solution are collectively referred to as aqueous glucose solution.

In the method for producing lactic acid of the present invention, lactic acid fermentation is continued by replacing a fermentation liquor created from an aqueous glucose solution with a high-K aqueous glucose solution, and such replacement of the fermentation liquor with the high-K aqueous solution is carried out at least once.

In order to increase the efficiency of the lactic acid fermentation, it is preferred that the lactic acid fermentation is continued until the amount of lactic acid accumulated in the fermentation liquor is maximized and the amount of glucose remained therein is minimized. The replacement of the fermentation liquor with a fresh aqueous glucose solution is preferably carried out, depending upon the glucose concentration at the time of starting fermentation, when the glucose concentration of the fermentation liquor becomes 10 g/L or lower. More preferably, the replacement is carried out when the glucose concentration of the fermentation liquor becomes 5 g/L or lower. However, in a case where the culturing time required for achieving the glucose concentration of the fermentation liquor of 10 g/L or lower is long, the replacement may be carried out at the glucose concentration higher than that.

As described above, when carrying out repetitive fermentation by using pre-grown fission yeast and a low-K aqueous glucose solution, the decrease in the lactic acid fermentation ability of the fission yeast may sometimes not be observed even after the fermentation liquor is replaced with the low-K aqueous glucose solution several times. The decrease in the lactic acid fermentation ability means that the glucose concentration of the fermentation liquor does not reach 10 g/L or lower, or it takes a long period of time for achieving the glucose concentration of 10 g/L or lower (e.g. five times longer comparing to a case where the lactic acid fermentation is not decreased).

Assuming that the lactic acid fermentation is carried out (n+1) times by repeatedly replacing with a low-K aqueous glucose solution from the first lactic acid fermentation (replacement with a low-K aqueous glucose solution is carried out n times), and the decrease in the lactic acid fermentation activity is observed at the (n+1)-th lactic acid fermentation (n is an integer of at least 1). The decrease in the lactic acid fermentation activity may be observed at the first replacement with a low-K aqueous glucose solution (i.e. n=1), and may be observed at the fourth lactic acid fermentation after the replacement with a low-K aqueous glucose solution is carried out three times (n=3). Depending upon the types of the fission yeast having a lactic acid fermentation ability, n is from 2 to 5 in many cases. Further, the definition of 1 time fermentation may be appropriately determined taking the production efficiency and economical efficiency into consideration, and is suitably a fermentation by which glucose contained in the aqueous glucose solution is consumed to some extent.

In the present invention, it is preferred that the replacement of the fermentation liquor with a high-K aqueous glucose solution is carried out at the time of, or earlier than, the n-th replacement. Assuming that the replacement with a high-K aqueous glucose solution is carried out at the m-th replacement, m is preferably an integer of equal to or smaller than n. m may be 0. That is, lactic acid fermentation may be carried out by using a high-K aqueous glucose solution from the first lactic acid fermentation which uses pre-grown fission yeast.

After the lactic acid fermentation which uses a high-K aqueous glucose solution, in a case where the lactic acid fermentation is carried out further by replacing the fermentation liquor, the culture broth to be used for the replacement of the fermentation liquor may be a high-K aqueous glucose solution or a low-K aqueous glucose solution. When the potassium component is accumulated in the cells by the lactic acid fermentation which uses a high-K aqueous glucose solution, the decrease in the lactic acid fermentation ability may not be observed even after the lactic acid fermentation is continued with a low-K aqueous glucose solution. However, if the lactic acid fermentation is continued further by continuing the replacement with a low-K aqueous glucose solution, it can be considered that the decrease in the lactic acid fermentation ability occurs since potassium component is gradually lost from the cells, in the same manner as in the case of continuing the first lactic acid fermentation. Accordingly, in the same manner as described above, the fermentation liquor is replaced with a high-K aqueous glucose solution before the lactic acid fermentation ability decreases.

In the present invention, the fermentation liquor is a fermentation liquor created from an aqueous glucose solution by lactic acid fermentation. The fermentation culture broth (i.e. an aqueous glucose solution), which replaces the fermentation liquor, may be a low-K aqueous glucose solution or a high-K aqueous glucose solution, as described above.

The first lactic acid fermentation which uses pre-grown fission yeast is preferably carried out in a low-K aqueous glucose solution. The efficiency of the lactic acid fermentation which uses a low-K aqueous glucose solution may sometimes be higher than that of the lactic acid fermentation which uses a high-K aqueous glucose solution. Further, the low-K aqueous glucose solution is more beneficial in view of the economical efficiency. Further, in the first lactic acid fermentation which uses pre-grown fission yeast, the fermentation efficiency may increase as the amount of inorganic nutrient components other than potassium decreases, like potassium.

Similarly, at the time of replacing the fermentation liquor, in a case where decrease in the lactic acid fermentation ability is unlikely to occur, the incubation broth to be used for the replacement is preferably a low-K aqueous glucose solution. Therefore, the fermentation liquor generated by using a high-K aqueous glucose solution may be replaced with a low-K aqueous glucose solution.

In the method for producing lactic acid of the present invention, the number of times for replacing the fermentation liquor with an aqueous glucose solution is not particularly limited. In order to maximize the amount of lactic acid produced by using a certain amount of the fission yeast having a lactic acid fermentation ability, it is preferred that the total amount of the fermentation liquor is increased by increasing the number of times for replacing the fermentation liquor. However, the number of times for replacing the fermentation liquor is not unlimited, and the fermentation efficiency may sometimes be decreased by causes, other than the ones associated with potassium ion, such as the decrease in the lactic acid fermentation ability and the decrease in the amount of the cells due to death of fission yeast. In the present invention, the number of times for replacing the fermentation liquor with an aqueous glucose solution is at least 1 time, preferably from 2 to 20 times, and more preferably from 8 to 12 times in view of the fermentation efficiency and economical efficiency.

The method for replacing the fermentation liquor is not limited to the above-described one in which almost the total amount of the fermentation liquor is replaced with a fresh aqueous glucose solution, and may be a method in which a part of the fermentation liquor is replaced with a fresh aqueous glucose solution successively or intermittently while continuing lactic acid fermentation. By using the above-described high-K aqueous glucose solution as a fresh aqueous glucose solution, the decrease in the lactic acid fermentation ability can be prevented. Unlike the case of replacing the total amount, in the method of replacing successively or intermittently, the potassium concentration of the whole culture broth does not reach 400 ppm or higher immediately after the partial replacement with a high-K aqueous glucose solution. However, when the amount of the high-K aqueous glucose solution for replacing is increased with time, the potassium ion concentration of the whole culture broth increases gradually, whereby the decrease in the lactic acid fermentation ability can be prevented. Further, even if it is a temporary, the potassium ion concentration of the whole culture broth in a fermentation tank is preferably at least 400 ppm.

In the method of replacing successively or intermittently, it is preferred to use a high-K aqueous glucose solution having a higher potassium ion concentration, or use a high-K aqueous glucose solution always as a fresh aqueous glucose solution. However, as described above, at least for the first lactic acid fermentation which uses pre-grown cells, a low-K aqueous glucose solution is preferably used.

Further, in the method of replacing successively or intermittently, the total amount of an aqueous glucose solution to be used for replacing the fermentation liquor is not particularly limited. However, as previously described, assuming that the case where the fermentation liquor is replaced with an aqueous glucose solution in an amount corresponds to the amount of the culture broth contained in the fermentation tank to be replaced with is set as 1 time replacement, the replacement time is at least 1 time, preferably from 2 to 100 times, and in view of the fermentation efficiency and economic efficiency, from 10 to 50 times is more preferred.

In order to increase the efficiency of the lactic acid fermentation, it is preferred to suppress the growth of fission yeast during the lactic acid fermentation, and the growth rate of the fission yeast at a temperature of 30° C. as represented by the following equation is preferably at most 1.5.

Growth rate=(dried cell weight after fermentation for 7 hours)/(dried cell weight at the time of starting fermentation)

In the above equation, the dried cell weight after fermentation means a dried cell weight after fermentation per 1 L of a growing culture broth, fermentation culture broth, or fermentation liquor (g dried cell-weight/L).

Further, during culturing for growing fission yeast, the growth rate of the fission yeast as represented by the above equation is usually from 4 to 12.

In the present invention, it is preferred to collect cells grown by culturing the fission yeast having a lactic acid fermentation ability in a liquid culture broth, and carry out lactic acid fermentation by using the collected cells. That is, when starting lactic acid fermentation, to obtain a certain amount of fission yeasts to be used for the lactic acid fermentation, it is preferred to grow the fission yeast having a lactic acid fermentation ability. The culture for growing fission yeast is carried out by using a growing culture broth, and the fission yeast is grown in the broth to increase its cell number. After obtaining a certain amount of the cells by the culture for growing fission yeast, the growing culture broth is replaced with a fermentation culture broth (aqueous glucose solution), thereby to continue the lactic acid fermentation. Further, depending on the case, during carrying out lactic acid fermentation by replacing the fermentation liquor with an aqueous glucose solution, the fermentation liquor may be replaced with a growing culture broth to carry out culturing for growing fission yeast so as to increase the amount of cells, and then the growing culture broth is replaced with a fermentation culture broth (aqueous glucose solution) thereby to carry out lactic acid fermentation continuously.

Now, the present invention will be described in detail.

### [Fission Yeast]

The fission yeast having a lactic acid fermentation ability to be used in the present invention is a fission yeast (yeast of the genus Schizosaccharomyces) imparted with a lactic acid fermentation ability. Originally, fission yeasts do not have a lactic acid fermentation ability. On the other hand, fission yeasts have high resistance to acids and can survive even when the pH of the surrounding environment is around 2. Accordingly, by introducing a gene for lactic acid fermentation to a fission yeast to prepare a fission yeast having a lactic acid fermentation ability, and using the fission yeast, it becomes possible to produce lactic acid without requiring neutralization.

The fission yeast to be used as the host for introducing the gene may be a mutant-type in which a specific gene is deleted or inactivated depending on application. As the fission yeast, Schizosaccharomyces pombe, Schizosaccharomyces japonicus, and Schizosaccharomyces octosporus may, for example, be mentioned. Among the above-mentioned fission yeasts, Schizosaccharomyces pombe (hereinafter sometimes referred to as S. pombe) is preferred in view of the availability of various useful mutant-strains.

Further, the entire nucleotide sequence database of the chromosome of S. pombe is stored and opened to the public in the database “GeneDB” of Sanger Institute as “Schizosaccharomyces pombe Gene DB (http://www.genedb.org/genedb/pombe/)”. Therefore, the sequence data for genes of S. pombe are available from the database and searchable by the gene name and the above-mentioned systematic name.

As the fission yeast to be used as a host, one having a marker for selecting a transformant is preferred. For example, it is preferred to use a host which essentially requires a specific nutrient factor for growth due to deletion of a gene. When preparing a transformant by using a vector containing a desired gene sequence, by introducing the deleted gene (auxotrophic complementation marker) to the vector, a transformant lacking the auxotrophy of the host will be obtained. It is possible to select the transformant by using the difference in auxotrophy between the host and the transformant.

For example, a yeast of the genus Schizosaccharomyces host which has been made auxotrophic for uracil by deletion or inactivation of orotidine 5′-phosphate decarboxylase (ura4 gene) is transformed with a vector containing ura4 gene (auxotrophic complementation marker), and transformants carrying the vector are obtained by selecting ones lacking uracil auxotrophy. The gene to be deleted to make an auxotrophic host is not limited to ura4 gene when it is used for selection of a transformant, and may, for example, be isopropyl malate dehydrogenase gene (leu1 gene).

When the transformant obtained by using the above-described auxotrophic host or the like has an auxotrophy, to culture it, the required nutrient should be added to a growing culture broth or a fermentation culture broth to be used for lactic acid fermentation. However, the necessity of the use of a specific nutrient for the lactic acid fermentation culture broth may increase the costs for producing lactic acid. Therefore, when an auxotrophic transformant is obtained, it is preferred to eliminate its auxotrophy before using it for lactic acid fermentation. The elimination of auxotrophy may be carried out by publicly known methods. For example, a method of introducing the deleted gene or selecting mutants having no auxotrophy may be used for eliminating auxotrophy.

As the method for introducing a gene which is not intrinsic to fission yeast and obtaining a fission yeast transformant which can express the introduced gene, publicly known genetic engineering techniques may be used. As the method for introducing structural gene of a heterogeneous protein into S. pombe as the host, the methods described in JP-A-5-15380, WO95/09914, JP-A-10-234375, JP-A-2000-262284, JP-A-2005-198612 and WO2010/087344 may, for example, be used.

For the fission yeast having a lactic acid fermentation ability, when introducing a gene which imparts a lactic acid fermentation ability, it is preferred to delete or inactivate a gene which is intrinsic to fission yeast and may decrease or inhibit the lactic acid fermentation ability of the transformant having a lactic acid fermentation ability obtained by the gene introduction.

For deletion or inactivation of a specific gene, publicly known methods can be used. Specifically, the Latour system (Nucleic Acids Res. (2006) 34: ell, and WO2007/063919) can be used to delete the gene. Further, the gene can be inactivated by mutating the gene at a certain position by mutant screening using mutagens (Koubo Bunshi ldengaku Jikken-Hou, 1996, Japan Scientific Societies Press), random mutations using PCR (polymerase chain reaction) (PCR Methods Appl., 1992, vol. 2, p. 28-33) and the like. As the yeast of the genus Saccharomyces host in which a specific gene is deleted or inactivated, ones disclosed in WO2002/101038 and WO2007/015470 may, for example, be used.

As the fission yeast, since wild-type one does not have a lactic acid fermentation ability, a mutant or a transformant having a lactic acid fermentation ability is used. As the reason why wild-type fission yeasts do not have a lactic acid fermentation ability, the fact that they do not have a functional lactate dehydrogenase (LDH) gene may be mentioned. Therefore, it is preferred to use a fission yeast transformant having a gene encoding LDH derived from other organism (hereinafter referred to as LDH gene) in a chromosome or as an extrachromosomal gene. The LDH gene is not particularly limited, and it may, for example, be an LDH gene derived from the microorganisms belonging to the genus Bifidobacterium, the genus Lactobacillus and the like and an LDH gene derived from mammals such as human and the like. Among them, a mammal-derived LDH gene is preferred from the viewpoint of excellent efficiency of lactic acid production by S. pombe. Particularly, a transformant in which a gene encoding human-derived L-LDH is integrated into its chromosome is preferred.

In the fission yeast imparted with a lactic acid fermentation ability, pyruvic acid generated from glucose by the glycolytic system is reduced into lactic acid by the action of lactate dehydrogenase. On the other hand, in fission yeasts, essentially, pyruvic acid is converted into acetaldehyde by the action of pyruvate decarboxylase, and then reduced into ethanol by the action of an alcohol dehydrogenase. That is, fission yeasts produce ethanol essentially by an alcohol fermentation.

In the present invention which intends to produce lactic acid, if the amount of pyruvic acid consumed by an alcohol fermentation becomes large, the proportion of the lactic acid obtained from glucose decreases, whereby the efficiency of lactic acid fermentation decreases. Accordingly, it is preferred to suppress the alcohol fermentation in order to increase the efficiency of lactic acid fermentation.

The present inventors tried to increase the efficiency of lactic acid fermentation of the fission yeast imparted with a lactic acid fermentation ability, by deleting or inactivating a gene encoding pyruvate decarboxylase.

For the gene encoding pyruvate decarboxylase (pyruvate decarboxylase gene, hereinafter referred to as “pdc gene”) in S. pombe, 4 types of groups comprised of a gene encoding pyruvate decarboxylase 1 (hereinafter referred to as “pdc 1 gene”), a gene encoding pyruvate decarboxylase 2 (hereinafter referred to as “pdc 2 gene”), a gene encoding pyruvate decarboxylase 3 (hereinafter referred to as “pdc 3 gene”), and a gene encoding pyruvate decarboxylase 4 (hereinafter referred to as “pdc 4 gene”) are known. Among them, pdc 2 gene and pdc 4 gene are the pdc genes which have major functions in S. pombe. Systematic names of the respective PDC genes are as follows.

pdc 1 gene (Pdc 1): SPAC13A11.06

pdc 2 gene (Pdc 2): SPAC1F8.07c

pdc 3 gene (Pdc 3): SPAC186.09

pdc 4 gene (Pdc 4): SPAC3G9.11c

As the pdc gene to be deleted or inactivated, pdc 2 gene is particularly preferred. The pdc 2 gene is a pdc gene which has a particularly major function.

If all the-described pdc genes are deleted or inactivated, the growth of the transformant is inhibited because it cannot carry out the ethanol fermentation. Therefore, deletion of inactivation of the pdc genes should be carried out in such a manner that an ethanol fermentation ability necessary for the growth is maintained so that sufficient amount of the transformant can be obtained and also that the ethanol fermentation ability is lowered so that the fermentation efficiency of lactic acid is improved. The present inventors have carried out an examination on this problem and found as a result that when pdc 2 gene is deleted or inactivated, pdc 4 gene is activated to a certain degree, whereby it becomes possible to attain both the ethanol fermentation ability for obtaining sufficient amount of the transformant and the production of lactic acid at a high fermentation efficiency (refer to the specification of PCT/JP2010/063888).

As described above, the fission yeast having a lactic acid fermentation ability to be used in the present invention is particularly preferably a transformant of Schizosaccharomyces pombe in which human-derived L-LDH gene is integrated into its chromosome and pdc2 gene is deleted or inactivated.

### [Lactic Acid Fermentation and Growth]

Lactic acid fermentation is one type of fermentation which produces lactic acid via pyruvic acid by using glucose as a starting material. The fission yeast having a lactic acid fermentation ability of the present invention can carry out lactic acid fermentation even in aerobic environments.

In the present invention, lactic acid fermentation is carried out in an aqueous glucose solution. The lactic acid fermentation is carried out by incubating (culturing) the fission yeast having a lactic acid fermentation ability in an aqueous glucose solution. The cultivation temperature is preferably from 20 to 37° C., more preferably from 28 to 32° C. Since the fission yeast will be precipitated when it is left to stand still, the lactic acid fermentation is preferably carried out with stirring or shaking. There is no particular limitation about the types of cultivation vessel and stirring-shaking apparatus, and publicly known ones may be appropriately selected for use.

In the lactic acid fermentation, the amount of the cells of fission yeast in the aqueous glucose solution is preferably from 18 to 72 g dried cell-weight/L.

During the cultivation in an aqueous glucose solution, since nutrients other than a carbon source are scarcely contained therein, the growth of fission yeast is poor comparing to the case where cultivation is carried out in a yeast culture broth such as YPD or SC. That is to say, in order to increase the efficiency of lactic acid fermentation, a culture broth containing nutrient sources other than a carbon source (particularly a nitrogen source) in a small amount may be used as the aqueous glucose solution so as to decrease the growth rate. As described above, the growth rate of the fission yeast as represented by the above equation is preferably at most 1.5.

### [Aqueous Glucose Solution]

The aqueous glucose solution to be used in the present invention (high-K aqueous glucose solution and low-K aqueous glucose solution) as a fermentation culture broth is one prepared by dissolving glucose in water. The content of glucose is preferably from 30 to 200 g/L, more preferably from 50 to 150 g/L.

The aqueous glucose solution to be used in the present invention is not a culture broth for growing fission yeast, and is one for lactic acid fermentation. Therefore, except for the presence of potassium ion, a component other than glucose like a metal ion or a trace nutrient source such as vitamins may be contained therein, but it is preferred that components not essential for lactic acid fermentation are not included therein as much as possible so that the step of recovering lactic acid from a lactic acid fermentation liquor generated by the fermentation by fission yeast becomes simple.

Particularly, the nitrogen source is, while it is a component largely contained a culture broth for growing yeasts, not essential for lactic acid fermentation. Accordingly, the content of the nitrogen source in the aqueous glucose solution to be used in the present invention is preferably at most 0.5 g/L, more preferably from 0 to 0.3 g/L. The nitrogen source content of from 0 to 0.3 g/L means that the nitrogen source is not contained or contained in an amount of at most 0.3 g/L.

In the present invention, the nitrogen source is a molecule containing a nitrogen atom which can be utilized by the fission yeast, and may, for example, be an amino acid such as glycine or alanine, a purine nucleobase such as adenine or guanine, a pyrimidine nucleobase such as cytosine, thymine or uracil, a nucleoside, a nucleotide, a ribonucleotide, a deoxyribonucleotide, DNA, RNA, a peptide, a polypeptide, ammonia, an ammonium ion (NH₄⁺ ion) derived from an ammonium salt such as ammonium sulfate, ammonium carbonate, ammonium chloride, ammonium phosphate, or ammonium acetate, an amine such as urea or triethylamine, a nitrate ion (NO₃⁻) derived from a nitrate salt such as aluminum nitrate, iron nitrate, or magnesium nitrate, and a nitrite ion (NO₂⁺) derived from a nitrite salt. In the aqueous glucose solution, the total content of the nitrogen sources is preferably from 0 to 0.3 g/L.

The below-described trace nutrient source such as vitamins may be included as the nitrogen source so long as it contains a nitrogen atom. Further, the nitrate ion derived from potassium nitrate may be included as the nitrogen source. However, if the amount of the nitrogen source exceeds the below-described range as a result of using a large amount of potassium nitrate so as to achieve the required concentration of potassium ion, it is preferred that potassium nitrate is not used or used in combination with other potassium sources so that the amount of the nitrogen source falls within the above-described range.

Further, the above-described preferred content of the nitrogen source is a value of before starting lactic acid fermentation. The components derived from the cells of fission yeast died and decomposed in the process of lactic acid fermentation are not included.

### (Potassium Ion)

The potassium compound used as the potassium ion source is a compound generates a potassium ion when it is dissolved in water, and is preferably a water-soluble inorganic potassium compound (such as an inorganic potassium salt) or a potassium salt of an organic acid. For example, a potassium salt such as potassium hydroxide, potassium carbonate, potassium hydrogen tartrate, potassium hydrogen carbonate, potassium chloride, potassium acetate, potassium sulfate, potassium nitrate, potassium nitrite, potassium dihydrogen phosphate, dipotassium hydrogen phosphate, tripotassium phosphate, potassium chlorate, or potassium perchlorate may be mentioned. The water-soluble inorganic potassium compound is more preferred, and a potassium halide such as potassium chloride is particularly preferred.

As described above, the potassium ion concentration of the high-K aqueous glucose solution is at least 400 ppm, more preferably from 400 to 4,000 ppm. The potassium ion concentration of the low-K aqueous glucose solution is less than 400 ppm, and may be 0 ppm. The low-K aqueous glucose solution is preferably an aqueous glucose solution having a potassium ion concentration of from 0 to 200 ppm, more preferably an aqueous glucose solution having a potassium ion concentration of from 0 to 100 ppm.

### (Alkali Metal Ions and Alkali Earth Metal Ions)

The aqueous glucose solution to be used in the present invention may contain at least one type of a metal ion selected from the group consisting of alkali metal ions other than a potassium ion and alkali earth metal ions.

As the alkali metals, lithium, sodium and rubidium may, for example, be mentioned. Among them, lithium and sodium are preferred. The total content of the alkali metals other than potassium in the aqueous glucose solution is preferably from 0 to 900 ppm, more preferably from 0 to 100 ppm.

As the alkali earth metals, beryllium, magnesium, calcium, strontium and barium may, for example, be mentioned. Among them, magnesium and calcium are preferred. The total content of the alkali earth metals in the aqueous glucose solution is preferably from 0 to 900 ppm, more preferably from 0 to 200 ppm.

The alkali metals and alkali earth metals are contained in the aqueous glucose solution in the form of ions. In a case where counterions contain nitrogen atoms, such counterions containing nitrogen atoms are included in the above-described nitrogen source.

### (Trace Metals)

The aqueous glucose solution to be used in the present invention preferably does not contain a part or all of the ions of metals which are other than alkali metals and alkali earth metals and are required for the growth of fission yeast, or does not contain them in an amount required for the growth of fission yeast.

As the metals required for the growth of fission yeast, iron which is the main element, and boron, aluminum, silicon, vanadium, chromium, manganese, cobalt, nickel, copper, zinc, arsenic, selenium and molybdenum which are trace elements, may be mentioned.

For example, the amount required for the growth of fission yeast is, in the case of S. pombe transformants, per 1 L of a culture broth, at least 145 mg as H₃BO₃, at least 155 mg as MnCl₂, at least 20 mg as CoCl₂.6H₂O, at least 22 mg as NiSO₄.6H₂O, at least 190 mg as CuSO₄.5H₂O, and at least 1,270 mg as ZnSO₄.7H₂O. Therefore, in the case of adding these compounds to the aqueous glucose solution, the amounts of these compounds are preferably lower than the above-described required amounts.

### (Trace Nutrient Source)

The aqueous glucose solution to be used in the present invention may contain a trace nutrient source such as vitamins. As the vitamins, biotin, pantothenic acid, nicotinic acid and inositol may, for example, be mentioned. The content of the trace nutrient source in the aqueous glucose solution is preferably from 0 to 300 ppm.

### [Replacement]

In the present invention, the replacement of the fermentation liquor with an aqueous glucose solution means that the fermentation liquor containing the cells of fission yeasts generated by lactic acid fermentation is recovered to separate a fermentation liquor, and then an aqueous glucose solution is newly supplied to the cells. The method for recovering the fermentation liquor may be any method, and a method of recovering the supernatant after the fermentation liquor is left to stand still and the cells are precipitated, a method of separating the fermentation liquor from the cells by using a filtration apparatus such as a filter, and a method of recovering the supernatant after precipitating the cells by centrifugation may, for example, be mentioned. Further, in the case of continuing the lactic acid fermentation after growing the cells, the growing culture broth is removed from the growing culture broth used for growing the cells by the above-described method, and then an aqueous glucose solution is supplied to the cells to carry out lactic acid fermentation.

### [Growth]

The fission yeast having a lactic acid fermentation ability to be used in the present invention may be one stored by freezing or scraped off from an agar plate, and can be used for lactic acid fermentation by suspending it to an aqueous glucose solution. However, when performing mass production of lactic acid, it is preferred to grow the fission yeast as the seed culture firstly, and separate the grown cells from the growing culture broth to collect the cells, and then carry out lactic acid fermentation by using the collected cells. As the method for collecting the grown cells from the growing culture broth, in the same manner as described above, a method of recovering the supernatant after the growing culture broth is left to stand still and the cells are precipitated, a method of separating the growing culture broth from the cells by using a filtration apparatus such as a filter, and a method of recovering the supernatant after precipitating the cells by centrifugation may, for example, be mentioned.

The growing culture broth may be a publicly know culture broth so long as it can grow the fission yeast having a lactic acid fermentation ability, and one prepared by adding essential amino acids or nucleic acids to a culture broth such as YPD, YPED, SC or SD medium, and EMM may, for example, be mentioned as the growing culture broth. The composition of the culture broth may, for example, be one described in the homepage of the Forsburg laboratory, University of Southern California (http://www-bcf.usc.edu/˜forsburg/media.html), and one described in Methods in Yeast Genetics, A Cold Spring Harbor Laboratory Course Manual, 2005 Edition, published by Cold Spring Harbor Laboratory Press.

### [Fermentation Activator]

The present invention further relates to a fermentation activator comprised of a potassium ion source.

The fermentation activator of the present invention is an additive to be added to an aqueous glucose solution which is not intended for the growth of fission yeast and is mainly used for a lactic acid fermentation by a fission yeast having a lactic acid fermentation ability to prevent the decrease in the lactic acid fermentation ability of the fission yeast. The aqueous glucose solution which is not intended for the growth of fission yeast and is mainly used for lactic acid fermentation is an aqueous glucose solution having a nitrogen source content of at most 0.3 g/L. Further, the fermentation activator is comprised of the above-described water-soluble potassium compound which can generate a potassium ion.

The fermentation activator of the present invention may preliminarily be added to the aqueous glucose solution for lactic acid fermentation in an amount such that the potassium ion concentration is at least 400 ppm. Further, in addition, it may be added to a culture broth during the lactic acid fermentation when the decrease in lactic acid fermentation ability is likely to occur or the decrease in lactic acid fermentation ability is observed.

The water-soluble potassium compound used as the fermentation activator is preferably a water-soluble inorganic potassium compound (such as an inorganic potassium salt) or a potassium salt of an organic acid. As the fermentation activator, the water-soluble inorganic potassium compound is preferred, and a potassium halide such as potassium chloride is particularly preferred. The formulation is not particularly limited and may, for example, be a powder or a tablet, and may be used as an aqueous solution.

### Examples

Now, the present invention will be described in further detail with reference to specific Examples. However, the present invention is not restricted by the following descriptions. In the following Examples, the term “%” means “mass %” unless otherwise noted.

### [Preparation of a Fission Yeast Having a Lactic Acid Fermentation Ability]

A strain restored with leu1 mutation of the fission yeast having a lactic acid fermentation ability prepared in Examples described in the specification of International Application No. PCT/JP2010/063888 was used. Such a fission yeast was prepared by the following method.

### <Preparation of Pdc2 (Systematic Name: SPAC1F8.07c) Deletion Strain>

A uracil-auxotrophic strain of S. pombe (ARC010, genotype: h-leu1-32 ura4-D18, provided from professor Yuichi lino, Molecular genetics research laboratory, Graduate school of science, The university of Tokyo) was transformed in accordance with the Latour method to prepare a deletion strain in which a gene encoding pyruvate decarboxylase (PDC) was deleted. For the preparation of deletion fragments, the whole genomic DNA prepared from ARCO32 strain of S. pombe (genotype: h−, provided from Professor Yuichi lino, Molecular genetics research laboratory, Graduate school of science, The university of Tokyo) by using DNeasy (manufactured by QIAGEN) was used as the template, and the 8 types of synthetic oligo-DNA (manufactured by Operon) having the below-listed sequences were used for pdc2 gene to be deleted.

UF:
5′-CTCTCCAGCTCCATCCATAAG-3′
UR:
5′-GACACAACTTCCTACCAAAAAGCCTTTCTGCCCATGTTTTC
TGTC-3′
OF:
5′-GCTTTTTGGTAGGAAGTTGTGTC-3′
OR:
5′-AGTGGGATTTGTAGCTAAGCTGTATCCATTTCAGCCGTTTGTG-3′
DF:
5′-AAGTTTCGTCAATATCACAAGCTGACAGAAAACATGGGCAG
AAAG-3′
DR:
5′-GTTCCTTAGAAAAAGCAACTTTGG-3′
FF:
5′-CATAAGCTTGCCACCACTTC-3′
FR:
5′-GAAAAAGCAACTTTGGTATTCTGC-3′

Each of UP region, OL region, and DN region was prepared by a PCR amplification of using KOD-Dash (manufactured by Toyobo Co. Ltd.) with UF and UR, OF and OR, and DF and DR, respectively. Then, using these regions as respective templates, full-length deletion fragments were prepared by a similar PCR amplification of using FF and FR. At the time of preparing the full-length deletion fragments, the below-listed two types of synthetic oligo-DNA (manufactured by Operon) were used, the whole genomic DNA similarly prepared from ACR 032 strain was used as a template, and a ura4 region fragment prepared by a similar PCR amplification was also used as a template.

5′-AGCTTAGCTACAAATCCCACT-3′
5′-AGCTTGTGATATTGACGAAACTT-3′

The deletion strain prepared by using thus obtained pdc2 gene deletion fragments was named IGF543. By using a culture broth containing 5-fluoroorotic acid (5-FOA), ura4-strain was selected from IGF543 strain (the name IGF543 was succeeded).

Thereafter, in order to increase the growth rate, IGF543 strain was streaked on YES plate (yeast extract 0.5%/glucose 3%/SP supplement) and cultured at 25° C., and then thus obtained colonies were sub-cultured in YPD medium (yeast extract 1%/peptone 2%/glucose 2%), and cultured at 25° C. Then, by using a culture broth having sufficiently grown cells, a glycerol stock was prepared and preserved at −80° C. The above-mentioned procedure was repeated until an appropriate growth rate was obtained, and a strain showing high growth rate was selected (the name IGF543 was succeeded).

<Preparation of a S. pombe Stain Producing Lactate Dehydrogenase>
(Preparation of pTL2HsLDH-Tf2)

A gene fragment encoding human L-lactate dehydrogenase structural gene (HsLDH-ORF) described in a reference (Tsujibo et al., Eur. J. Biochem., 1985, vol. 147, pp. 9-15) was amplified by PCR using a human fibroblast cDNA library introduced into Okayama vector as a template and using the following primer set having a restriction enzyme NcoI recognition sequence at the 5′ end side and a restriction enzyme SaII recognition sequence at the 3′ end side:

(No. 4620)
5′-GTCCATGGCAACTCTAAAGGATCAG-3′,
(No. 4621)
5′-CAGTCGACTTAAAATTGCAGCTCCTTTTG-3′

Thus obtained amplified fragments were double-digested using restriction enzymes NcoI and SaII and then incorporated between AfIIII and SaII of multi-cloning vector pTL2M5 described in JP-A-2000-262284, thereby to obtain LDH expression vector pTL2HsLDH.

The pTL2HsLDH was double-digested with restriction enzymes Sepl and Bst1107I, and then thus obtained fragments (hCMV promoter/LDH-ORF/LPI terminator) were inserted between the recognition sequences sites for restriction enzymes NheI and KpnI (blunt-ended) of Tf2 multilocus integration type vector pTf2MCS-ura4 prepared by the following process, thereby to obtain integration type L-lactate dehydrogenase gene expression vector pTL2HsLDH-Tf2. Further, with regard to the method for introducing genes into Tf2 transposon gene loci, refer to WO2010/087344.

(Preparation of pTf2MCS-ura4)

Preparation process of pTf2MCS-ura4 is as follows. That is, the whole genomic DNA of S. pombe was purified from cells by using a whole genomic DNA extraction kit (DNeasy, manufactured by QIAGEN), and then a Tf2-2 (systematic name: SPAC167.08 gene available from GeneDB) DNA fragment (about 3,950 bp) of S. pombe was amplified by a PCR amplification of using 1 μg of the DNA as a template, and the following primer pair in which a restriction enzyme BsiWI recognition sequence (CGTACG) was introduced into the 5′ end side:

5′-AAGGCCTCGTACGTGAAAGCAAGAGCAAAACGA-3′,
5′-AAGGCCTCGTACGTGCTTTGTCCGCTTGTAGC-3′

The both ends of thus amplified DNA fragments were treated with a restriction enzyme BsiWI, and then separation and purification were carried out by agarose gel electrophoresis to prepare an insert fragment.

Then, the chromosomal integration vector pXL4 (Idiris et al., Yeast, Vol. 23, pp. 83-99, 2006) was digested with the same restriction enzyme BsiWI to prepare a region (about 2,130 bp) containing an ampicillin resistant gene (ApR) and a replication origin of E. coli (pBR 322 ori). The DNA fragment was further treated with a dephosphorylase (CIAP, manufactured by Takara Bio Co., Ltd.) for dephosphorylation, and then separated and purified by agarose gel electrophoresis to prepare a vector fragment.

Ligation of the insert fragment and the vector fragment was carried out by using a ligation kit (DNA Ligation Kit ver. 2, manufactured by Takara Bio Co., Ltd.), followed by transformation of E. coil DH5 (Toyobo Co., Ltd.) to prepare recombination plasmid pTf2-2 (6,071 bp).

By using 0.1 μg of the above-constructed vector pTf2-2 as a template and a primer pair comprised of a primer 5′-GGGGTACCAAGCTTCTAGAGTCGACTCCGGTGCTACGACACTTT-3′ (which has recognition sequences for KpnI, HindIII, XbaI and SaII at the 5′ end) and a primer 5′-GGGGTACCAGGCCTCTCGAGGCTAGCCATTTCCAGCGTACATCCT-3′ (which has recognition sequences for KpnI, StuI, XhoI and NheI at the 5′ end), a PCR amplification was carried out to obtain fragments having the whole length of 6,060 bp. After KpnI digestion of the both ends, the fragments were separated and purified by agarose gel electrophoresis, followed by self ligation by using a ligation kit to prepare pTf2 (MCS) vector having a length of 6,058 bp and a multiple cloning site (MCS) within the nucleotide sequence of Tf2-2 retrotransposon.

The above-constructed pTf2 (MCS) vector was double-digested with restriction enzymes KpnI and NheI, and then separated and purified by agarose gel electrophoresis to prepare a 6,040-bp fragment. Further, a fragment having recognition sequences for restriction enzymes KpnI and NheI at both ends of S. pombe uracil-auxotrophy marker ura4 (orotidine-5′-phosphate decarboxylase gene, GeneDB systematic name: SPCC330.05c) which were introduced by using PCR, was prepared, and double-digested with restriction enzymes KpnI and NheI, and then separated and purified by agarose gel electrophoresis to obtain a 2,206-bp fragment. The two fragments obtained were ligated by using a ligation kit to prepare pTf2 (MCS)-ura4 vector having a length of 8,246 bp and a multiple cloning site (MCS) within the nucleotide sequence of Tf2-2 retrotransposon.

### (Transformation and Strain Selection)

By using the above-prepared vector pTL2HsLDH-Tf2, IGF543 strain (growth rate-recovered strain) was transformed by the method of Okazaki et al. (Okazaki et al., Nucleic Acids Res., 1990, vol. 18, pp. 6485-6489) and spread on a selection medium MMA+Leu plate.

Each of a large number of thus obtained single colonies was inoculated in YPD16 medium (yeast extract 1%/peptone 2%/glucose 16%) and cultured at 32° C. for 72 hours, and then, using the culture supernatant alone as a sample, the concentrations of glucose, ethanol and L-lactic acid and the pH of the medium were measured by using BF-4 and BF-5 (Oji Scientific Instruments). Based on the obtained results, those having high lactic acid productivity were selected again, and then cultured (20 hours, 44 hours, 66.5 hours, 80 hours, 176 hours) further in YPD12 medium (yeast extract 1%/peptone 2%/glucose 12%). Thereafter, the concentrations of glucose, ethanol and L-lactic acid and the pH of the medium were measured in the same manner, thereby to select a strain having the highest productivity of L-lactic acid. Thus selected strain was named ASP2782 (genotype: h⁻ leu1-32 ura4-D18 pdc2-D23 Tf2<HsLDH-ORF/ura4+).

### (Preparation of Leul Mutation Recovered Strain)

Integration type vector pXL4 for fission yeast (Idiris et al., Yeast, 2006, vol. 23, pp. 83-99) was double-digested with restriction enzymes, and thus obtained fragments were blunt-ended, followed by ligation to obtain expression vector pXL1(delta-neo) for fission yeast.

ASP2782 strain was transformed by using the pXL1(delta-neo) vector in accordance with the above-mentioned method of Okazaki et al., and then spread on a selection medium MMA plate. Each of the obtained single colonies was, as a leu1 mutation recovered strain, named ASP3054 (genotype: h⁻ leu1-32 ura4-D18 pdc2-D23 Tf2 <HsLDH-ORF/ura4+leu1+).

### Experiment Example 1

### Repetitive Culture in YD10 Medium or a Potassium Ion-Containing Aqueous Glucose Solution

The transformant of Schizosaccharomyces pombe (ASP3054 strain) in which Pdc2 was deleted and human-derived L-LDH gene was integrated into its chromosome was inoculated in 5 ml of D10 liquid medium (an aqueous solution that contains only 10% of glucose) to a concentration of about 30 g (on the dry cell weight basis)/L and cultured under conditions of a temperature of 30° C. and a stirring speed of 110 rpm, and then the concentrations of lactic acid and ethanol in the culture medium were measured (Table 1, 1^{st} time).

After completion of the culturing, culture supernatant and cells were recovered by centrifugation (6,000×g, 20 minutes).

Then, the recovered cells were added to YD10 liquid medium (yeast extract 1%, glucose 1%) or a potassium ion-containing aqueous glucose solution (Na₂HPO₄ 2.2 g/L, MgCl₂.6H₂O 1.05 g/L, CaCl₂.2H₂O 0.015 g/L, KCl 1 g/L, NaSO₄ 2.2 g/L, glucose 10%). A series of these operations was carried out 9 times (2^{nd} time to 10^{th} time).

The culturing time, the measurement results of the concentrations of glucose, ethanol, and lactic acid at the time of the completion of the culturing, and the sugar base yield of lactic acid obtained from the measurement results, obtained after culturing 10 times in total, are shown in Table 1.

TABLE 1

| | | Culturing | Concentration | Concentration | Concentration | Sugar base yield |
|---|---|---|---|---|---|---|
| | | time | of glucose | of ethanol | of lactic acid | of lactic acid |
| | | (hr) | (g/L) | (g/L) | (g/L) | (%) |
| YD10 medium | 1^{st} time | 4.3 | 0.9 | 15.4 | 72.6 | 73.2 |
| | 2^{nd} time | 4.8 | 1.8 | 16.5 | 80.9 | 82.3 |
| | 3^{rd} time | 4.5 | 3.2 | 16.3 | 79.6 | 82.2 |
| | 4^{th} time | 5.0 | 3.7 | 14.4 | 82.0 | 85.2 |
| | 5^{th} time | 5.8 | 3.2 | 13.2 | 84.4 | 87.1 |
| | 6^{th} time | 6.3 | 2.9 | 12.0 | 84.9 | 87.4 |
| | 7^{th} time | 6.5 | 2.3 | 12.1 | 87.1 | 89.2 |
| | 8^{th} time | 7.0 | 3.4 | 12.6 | 85.9 | 88.9 |
| | 9^{th} time | 7.3 | 3.0 | 12.6 | 84.7 | 87.3 |
| | 10^{th} time | 7.5 | 2.7 | 13.3 | 85.1 | 87.5 |
| Potassium ion- | 1^{st} time | 4.3 | 0.9 | 15.4 | 72.6 | 73.2 |
| containing | 2^{nd} time | 5.0 | 3.3 | 14.8 | 78.5 | 81.2 |
| aqueous glucose | 3^{rd} time | 5.0 | 4.0 | 14.8 | 78.0 | 81.3 |
| solution | 4^{th} time | 5.5 | 3.3 | 14.3 | 79.9 | 82.6 |
| | 5^{th} time | 6.3 | 3.0 | 13.7 | 79.1 | 81.5 |
| | 6^{th} time | 6.8 | 3.5 | 13.6 | 80.0 | 82.8 |
| | 7^{th} time | 7.5 | 3.9 | 13.6 | 78.5 | 81.7 |
| | 8^{th} time | 9.0 | 3.6 | 13.6 | 79.4 | 82.3 |
| | 9^{th} time | 10.3 | 3.9 | 13.6 | 80.2 | 83.5 |
| | 10^{th} time | 11.5 | 4.1 | 13.6 | 79.9 | 83.4 |

As apparent from Table 1, in the repetitive culture using the potassium ion-containing aqueous glucose solution, the sugar base yield of lactic acid was maintained at high level even when the culturing was repeated, whereby it was confirmed that lactic acid can be produced stably with high productivity without requiring neutralization with an alkali.

### Experiment Example 2

### Repetitive Culture in D10 Medium

The ASP3054 strain was inoculated in 5 ml of D10 liquid medium (glucose 10%) to a concentration of about 30 g (on the dry cell weight basis)/L and cultured under conditions of a temperature of 30° C. and a stirring speed of 110 rpm, and then the concentrations of lactic acid and ethanol in the culture medium were measured (1^{st} time).

After completion of the culturing, culture supernatant and cells were recovered by centrifugation (6,000×g, 20 minutes).

Then, the recovered cells were added to the same liquid medium to culture them again. A series of these operations was carried out 2 times (2^{nd} time, and 3^{rd} time).

The culturing time, the measurement results of the concentrations of glucose, ethanol, and lactic acid at the time of the completion of the culturing, and the sugar base yield of lactic acid obtained from the measurement results, obtained after culturing 3 times in total, are shown in Table 2.

TABLE 2

| | | Culturing | Concentration | Concentration | Concentration | Sugar base yield |
|---|---|---|---|---|---|---|
| | | time | of glucose | of ethanol | of lactic acid | of lactic acid |
| | | (hr) | (g/L) | (g/L) | (g/L) | (%) |
| D10 liquid medium | 1^{st} time | 4.3 | 0.9 | 15.4 | 72.6 | 73.2 |
| | 2^{nd} time | 11.8 | 3.1 | 16.3 | 77.1 | 79.6 |
| | 3^{rd} time | 84.8 | 31.9 | 7.2 | 53.5 | 78.6 |

As apparent from Table 2, in the repetitive culture using the D10 liquid medium, the sugar base yield of lactic acid decreased significantly when the culturing was repeated, whereby it was confirmed that high and stable production of lactic acid cannot be achieved. Particularly, at the 3^{rd} time culturing, a large amount of residual glucose was observed even after 84.8 hours.

### Experiment Example 3

### Repetitive Culture in a Potassium Ion or Sodium Ion-Containing Aqueous Glucose Solution

### (Growth)

The ASP3054 strain was inoculated in 5 ml of YPD10 liquid medium (yeast extract 1%, peptone 2%, glucose 10%) to a concentration of about 30 g (on the dry cell weight basis)/L and cultured under conditions of a temperature of 30° C. and a stirring speed of 110 rpm, and then the concentrations of lactic acid and ethanol in the culture medium were measured (growth). After completion of the culturing, culture supernatant and cells were recovered by centrifugation (6,000×g, 20 minutes). A series of these operations was carried out 2 times (growth 1, and growth 2).

### (Lactic Acid Fermentation)

The recovered cells were added to a potassium ion-containing aqueous glucose solution (potassium chloride 20 mM, glucose 10%) or a sodium ion-containing aqueous glucose solution (sodium chloride 20 mM, glucose 10%) to culture them. After completion of the culturing, cells were recovered by centrifugation, and then added to a fresh potassium ion-containing aqueous glucose solution or sodium ion-containing aqueous glucose solution. A series of these operations was carried out 2 times (1^{st} time and 2^{nd} time) for the potassium ion-containing aqueous glucose solution. On the other hand, for the sodium ion-containing aqueous glucose solution, a series of these operations was carried out once (1^{st} time).

The culturing time, the measurement results of the concentrations of glucose, ethanol, and lactic acid at the time of the completion of the culturing, and the sugar base yield of lactic acid obtained from the measurement results, obtained after the above-described culturing, are shown in Table 3.

TABLE 3

| | | Culturing | Concentration | Concentration | Concentration | Sugar base yield |
|---|---|---|---|---|---|---|
| | | time | of glucose | of ethanol | of lactic acid | of lactic acid |
| | | (hr) | (g/L) | (g/L) | (g/L) | (%) |
| K medium | Growth 1 | 6.5 | 0.0 | 17.0 | 73.5 | 73.5 |
| | Growth 2 | 7.5 | 0.0 | 17.4 | 82.3 | 82.3 |
| | 1^{st} time | 11.0 | 1.1 | 14.5 | 82.1 | 83.0 |
| | 2^{nd} time | 15.0 | 1.3 | 19.2 | 76.9 | 77.9 |
| Na medium | Growth 1 | 6.5 | 0.0 | 17.0 | 73.5 | 73.5 |
| | Growth 2 | 7.5 | 0.0 | 17.4 | 82.3 | 82.3 |
| | 1^{st} time | 108.0 | 9.7 | 9.3 | 68.4 | 75.7 |

As apparent from Table 3, in the repetitive culture using the potassium ion-containing aqueous glucose solution, the sugar base yield of lactic acid was maintained at high level even when the culturing was repeated. However, in the repetitive culture using the sodium ion-containing aqueous glucose solution, the production rate of lactic acid decreased significantly, whereby it was confirmed that high and stable production of lactic acid cannot be achieved. Particularly, in the culture using the sodium ion-containing aqueous glucose solution, a large amount of residual glucose was observed even after 108 hours.

### Experiment Example 4

### Growth Rate During Lactic Acid Fermentation

The ASP3054 strain was inoculated in YPD10 liquid medium (yeast extract 1%, peptone 2%, glucose 10%) to a concentration of about 30 g (on the dry cell weight basis)/L, and cultured by using a 3 L jar fermenter under conditions of a temperature of 30° C. and a stirring speed of 500 rpm. After completion of the culturing, culture supernatant and cells were recovered by centrifugation (6,000×g, 20 minutes).

The recovered cells were added to D10 liquid medium (glucose 10%) or K medium (potassium ion-containing aqueous glucose solution; potassium chloride 20 mM, glucose 10%) to culture them. After completion of the culturing, cells were recovered by centrifugation, and then washed with distilled water. After washing, the cells were recovered by centrifugation, and allowed to stand for 24 hours at 110° C. After confirming that the cells were sufficiently dried, the dried cell weight (g dried cell-weight/L) was measured, and the growth rate was calculated by the following equation from the dried cell weight at the time of starting fermentation (0 hour) and after fermentation for 7 hours (7 hours).

Growth rate=(dried cell weight after fermentation for 7 hours)/(dried cell weight at the time of starting fermentation)

TABLE 4

| | Dried cell weight | | |
|---|---|---|---|
| | (g dried cell-weight/L) | | |
| | 0 hour | 7 hours | Growth rate |
| D10 liquid medium | 20.9 | 21.1 | 1.01 |
| K medium | 20.8 | 21.3 | 1.02 |

### INDUSTRIAL APPLICABILITY

The lactic acid obtained by the production method of the present invention can be used as a raw material of polylactic acid or the like. Polylactic acid itself, a polymer alloy made of polylactic acid and other resins, etc. are biodegradable, and can be used for various products as biodegradable plastics.

This application is a continuation of PCT Application No. PCT/JP2012/053709, filed on Feb. 16, 2012, which is based upon and claims the benefit of priority from Japanese Patent Application No. 2011-035165 filed on Feb. 21, 2011. The contents of those applications are incorporated herein by reference in its entirety.

What is claimed is:

1. A method for producing lactic acid, which comprises subjecting glucose to lactic acid fermentation by using a fission yeast having a lactic acid fermentation ability and recovering lactic acid produced thereby, characterized in that a fermentation liquor created from an aqueous glucose solution by lactic acid fermentation is replaced with an aqueous glucose solution having a potassium ion concentration of at least 400 ppm to continue the lactic acid fermentation, and the replacement of the fermentation liquor with the potassium ion-containing aqueous glucose solution is performed at least once.

2. The method for producing lactic acid according to claim 1, wherein a fermentation liquor created from the aqueous glucose solution having a potassium ion concentration of at least 400 ppm by lactic acid fermentation is replaced with an aqueous glucose solution having a potassium ion concentration of lower than 400 ppm at least once.

3. The method for producing lactic acid according to claim 1, wherein the growth rate of the fission yeast as represented by the following equation is at most 1.5.
Growth rate=(dried cell weight after fermentation for 7 hours)/(dried cell weight at the time of starting fermentation)

4. The method for producing lactic acid according to claim 1, wherein the aqueous glucose solution to be used for the lactic acid fermentation contains from 30 to 200 g/L of glucose.

5. The method for producing lactic acid according to claim 1, wherein the potassium ion concentration of the aqueous glucose solution having a potassium ion concentration of at least 400 ppm is at most 4,000 ppm.

6. The method for producing lactic acid according to claim 1, wherein the aqueous glucose solution to be used for the lactic acid fermentation contains at least one type of a metal ion selected from the group consisting of alkali metal ions other than a potassium ion and alkali earth metal ions.

7. The method for producing lactic acid according to claim 1, wherein the aqueous glucose solution to be used for the lactic acid fermentation has a nitrogen source content of from 0 to 3.0 g/L.

8. The method for producing lactic acid according to claim 1, wherein the aqueous glucose solution to be used for the lactic acid fermentation does not contain ions of metals which are other than alkali metals and alkali earth metals and which are required for the growth of fission yeast, or does not contain such ions in an amount necessary for the growth of fission yeast.

9. The method for producing lactic acid according to claim 1, wherein the aqueous glucose solution having a potassium ion concentration of at least 400 ppm contains from 50 to 150 g/L of glucose, from 400 to 4,000 ppm of potassium ion, at least one type of a metal ion selected from the group consisting of alkali metal ions other than a potassium ion and alkali earth metal ions, an anion which is a counter ion of the metal ion including potassium ion, from 0 to 300 ppm of a micronutrient source other than the above-mentioned ones, and from 0 to 300 ppm of a nitrogen source (when the anion and the micronutrient source contain nitrogen atoms, the amount of such nitrogen atoms is included).

10. The method for producing lactic acid according to claim 1, wherein the lactic acid fermentation is carried out by using cells which are collected after culturing and growing a fission yeast having a lactic acid fermentation ability in a liquid culture broth.

11. The method for producing lactic acid according to claim 10, wherein the first lactic acid fermentation using the grown cells is carried out by using an aqueous glucose solution containing from 30 to 200 g/L of glucose.

12. The method for producing lactic acid according to claim 11, wherein the aqueous glucose solution to be used for the first lactic acid fermentation does not have a potassium ion concentration of at least 400 ppm.

13. The method for producing lactic acid according to claim 1, wherein the fission yeast having a lactic acid fermentation ability is a transformant expressing a gene encoding a lactate dehydrogenase derived from an organism excluding fission yeast.

14. The method for producing lactic acid according to claim 1, wherein the fission yeast having a lactic acid fermentation ability is a transformant in which pdc2 gene of fission yeast is deleted or inactivated.

15. A fermentation activator for activating a lactic acid fermentation by a fission yeast having a lactic acid fermentation ability in an aqueous glucose solution having a nitrogen source content of at most 0.3 g/L, characterized in that it is comprised of a water-soluble potassium compound which can generate a potassium ion.


SEQUENCE LISTING (16 sequences)

SEQ ID NO: 1 - DNA, 21 bp, Artificial Sequence
  Description of Artificial Sequence Synthetic oligonucleotide
ctctccagctccatccataag

SEQ ID NO: 2 - DNA, 45 bp, Artificial Sequence
  Description of Artificial Sequence Synthetic oligonucleotide
gacacaacttcctaccaaaaagcctttctgcccatgttttctgtc

SEQ ID NO: 3 - DNA, 23 bp, Artificial Sequence
  Description of Artificial Sequence Synthetic oligonucleotide
gctttttggtaggaagttgtgtc

SEQ ID NO: 4 - DNA, 43 bp, Artificial Sequence
  Description of Artificial Sequence Synthetic oligonucleotide
agtgggatttgtagctaagctgtatccatttcagccgtttgtg

SEQ ID NO: 5 - DNA, 45 bp, Artificial Sequence
  Description of Artificial Sequence Synthetic oligonucleotide
aagtttcgtcaatatcacaagctgacagaaaacatgggcagaaag

SEQ ID NO: 6 - DNA, 24 bp, Artificial Sequence
  Description of Artificial Sequence Synthetic oligonucleotide
gttccttagaaaaagcaactttgg

SEQ ID NO: 7 - DNA, 20 bp, Artificial Sequence
  Description of Artificial Sequence Synthetic oligonucleotide
cataagcttgccaccacttc

SEQ ID NO: 8 - DNA, 24 bp, Artificial Sequence
  Description of Artificial Sequence Synthetic oligonucleotide
gaaaaagcaactttggtattctgc

SEQ ID NO: 9 - DNA, 21 bp, Artificial Sequence
  Description of Artificial Sequence Synthetic oligonucleotide
agcttagctacaaatcccact

SEQ ID NO: 10 - DNA, 23 bp, Artificial Sequence
  Description of Artificial Sequence Synthetic oligonucleotide
agcttgtgatattgacgaaactt

SEQ ID NO: 11 - DNA, 25 bp, Artificial Sequence
  Description of Artificial Sequence Synthetic primer
gtccatggcaactctaaaggatcag

SEQ ID NO: 12 - DNA, 29 bp, Artificial Sequence
  Description of Artificial Sequence Synthetic primer
cagtcgacttaaaattgcagctccttttg

SEQ ID NO: 13 - DNA, 33 bp, Artificial Sequence
  Description of Artificial Sequence Synthetic primer
aaggcctcgtacgtgaaagcaagagcaaaacga

SEQ ID NO: 14 - DNA, 32 bp, Artificial Sequence
  Description of Artificial Sequence Synthetic primer
aaggcctcgtacgtgctttgtccgcttgtagc

SEQ ID NO: 15 - DNA, 44 bp, Artificial Sequence
  Description of Artificial Sequence Synthetic primer
ggggtaccaagcttctagagtcgactccggtgctacgacacttt

SEQ ID NO: 16 - DNA, 45 bp, Artificial Sequence
  Description of Artificial Sequence Synthetic primer
ggggtaccaggcctctcgaggctagccatttccagcgtacatcct